(19) United States
(12) Patent Application Publication
(10) Pub. No.: US 2021/0145858 A1
(43) Pub. Date: May 20, 2021

(54) Compositions for Renal Disease

(71) Applicant: Rakhi Khanna, Lake Forest, IL (US)

(72) Inventor: Rakhi Khanna, Lake Forest, IL (US)

(21) Appl. No.: 17/031,142

(22) Filed: Sep. 24, 2020

Related U.S. Application Data
(60) Provisional application No. 62/923,551, filed on Nov. 15, 2019.

(51) Int. Cl. A61K 31/714; A61K 31/519; A61K 31/4415; A61K 31/455; A61K 31/197; A61K 31/4188; A61K 33/30; A61K 33/08; A61K 47/18; A61K 31/59; A61K 33/10

(52) U.S. Cl. CPC A61K 31/714; A61K 31/519; A61K 31/4415; A61K 31/455; A61K 31/197; A61K 33/10; A61K 33/30; A61K 33/08; A61K 47/18; A61K 31/59; A61K 31/4188

(57) ABSTRACT

The present disclosure relates to compositions comprising a vitamin B component; vitamin D; a magnesium component; water; and optionally a zinc component. In some embodiments, the vitamin B component includes one or more of: folic acid, vitamin B12, vitamin B6, vitamin B1, vitamin B3, and vitamin B5 (e.g., any two, three, four, five, or all six of folic acid, vitamin B12, vitamin B6, vitamin B1, vitamin B3, and vitamin B5). Such compositions are useful for providing, for example, vitamin B; vitamin D, and magnesium to dialysis patients and/or patients with advanced kidney disease that need to restrict their water intake.

### CROSS-REFERENCE TO RELATED APPLICATIONS

This application claims priority to U.S. Application Ser. No. 62/923,551, filed on Oct. 19, 2019, which is hereby incorporated by reference in its entirety.

### TECHNICAL FIELD

The present disclosure relates to compositions comprising a vitamin B component; vitamin D; a magnesium component; water; and optionally a zinc component. Such compositions are useful for providing, for example, vitamin B; vitamin D, and magnesium to dialysis patients and/or patients with advanced kidney disease that need to restrict their water intake.

### BACKGROUND

Chronic fluid overload is a frequent complication in dialysis patients that can lead to swelling, discomfort, hypertension, left ventricular hypertrophy, arterial stiffness, atherosclerosis uremic cardiomyopathy, and cardiovascular morbidity. To avoid fluid overload, fluids are typically restricted in dialysis patients. However, many patients find it difficult to take all of their oral medications and still abide by their fluid restrictions.

### SUMMARY

Provided herein are compositions comprising: a vitamin B component; vitamin D; a magnesium component; and water.

In some embodiments, the vitamin B component comprises one or more of: folic acid, vitamin B₁₂, vitamin B₆, vitamin B₁, vitamin B₃, and vitamin B₅. In some embodiments, the vitamin B component comprises one or more of: folic acid present in an amount of about 500 μg to about 1600 μg; vitamin B₁₂ present in an amount of about 1 μg to about 10 μg; vitamin B₆ present in an amount of about 10 mg to about 50 mg; vitamin B₁ present in an amount of about 1 mg to about 2 mg; vitamin B₃ present in an amount of about 15 mg to about 25 mg; vitamin B₅ present in an amount of about 5 mg to about 15 mg; and biotin present in an amount of about 50 mg to about 200 mg.

In some embodiments, the vitamin B component comprises folic acid present in an amount of about 1000 μg.

In some embodiments, the vitamin B component comprises vitamin B₁₂ present in an amount of about 6 μg.

In some embodiments, the vitamin B component comprises vitamin B₆ present in an amount of about 25 mg.

In some embodiments, the vitamin B component comprises vitamin B₁ present in an amount of about 1.5 mg.

In some embodiments, the vitamin B component comprises vitamin B₃ present in an amount of about 20 mg.

In some embodiments, the vitamin B component comprises vitamin B₅ present in an amount of about 10 mg.

In some embodiments, the vitamin B component comprises biotin present in an amount of about 100 mg.

In some embodiments, the vitamin B component comprises folic acid, vitamin B₁₂, vitamin B₆, vitamin B₁, vitamin B₃, and vitamin B₅.

In some embodiments, the vitamin D is present in an amount of about 4,000 IU to about 10,000 IU. In some embodiments, the vitamin D is present in an amount of about 5,000 IU.

In some embodiments, the magnesium component comprises magnesium carbonate, magnesium oxide, magnesium chloride, magnesium citrate, magnesium sulfate, magnesium gluconate, magnesium hydroxide, or a combination thereof. In some embodiments, the magnesium component comprises magnesium carbonate present in an amount of about 100 mg to about 200 mg; magnesium oxide present in an amount of about 200 mg to about 500 mg; or a combination thereof.

In some embodiments, the magnesium oxide present in an amount of about 400 mg.

In some embodiments, the composition comprises: folic acid present in an amount of about 1000 μg; vitamin D is present in an amount of about 5,000 IU; and magnesium carbonate present in an amount of about 100 mg to about 200 mg.

In some embodiments, the composition further comprises zinc. In some embodiments, the zinc is present in an amount of about 25 mg to about 100 mg. In some embodiments, the zinc is present in an amount of about 50 mg.

In some embodiments, the composition further comprises one or more phosphorous binders. In some embodiments, the one or more phosphorous binders comprise: sevelamer HCl, lanthanum carbonate, sucroferric oxyhydroxide, or ferric citrate.

In some embodiments, the one or more phosphorous binders comprises: sevelamer HCl present in an amount of about 2000 mg to about 8000 mg; lanthanum carbonate present in an amount of about 1000 mg to about 3500 mg; sucroferric oxyhydroxide present in an amount of about 1000 mg to about 3500 mg; ferric citrate present in an amount of about 550 mg to about 3000 mg; or a combination thereof.

In some embodiments, the sevelamer HCl is present in an amount of about 2400 mg. In some embodiments, the sevelamer HCl is present in an amount of about 4800 mg. In some embodiments, the sevelamer HCl is present in an amount of about 7200 mg.

In some embodiments, the lanthanum carbonate is present in an amount of about 1500 mg. In some embodiments, the lanthanum carbonate is present in an amount of about 3000 mg.

In some embodiments, the sucroferric oxyhydroxide is present in an amount of about 1500 mg. In some embodiments, the sucroferric oxyhydroxide is present in an amount of about 3000 mg.

In some embodiments, the ferric citrate present in an amount of about 630 mg. In some embodiments, the ferric citrate present in an amount of about 1240 mg. In some embodiments, the ferric citrate present in an amount of about 1890 mg. In some embodiments, the ferric citrate present in an amount of about 2520 mg.

In some embodiments, the composition does not comprise one or more of: a protein component, vitamin C, vitamin A, vitamin K, vitamin E, iron, calcium, and an omega-3 fatty acid. In some embodiments, the composition does not comprise: a protein, vitamin C, vitamin A, vitamin K, vitamin E, calcium, and an omega-3 fatty acid.

In some embodiments, the composition further comprises a buffer.

In some embodiments, the composition does not comprise sodium chloride.

In some embodiments, the composition does not include a sugar or sweetener.

Also provided herein are methods for reducing overhydration in a patient in need thereof, the method comprising administering any of the compositions described herein.

In some embodiments, the composition is administered once daily. As used herein, “subject” or “patient” refers to any subject, particularly a mammalian subject, for whom diagnosis, prognosis, or therapy is desired, for example, a human.

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, databases entries, and other references mentioned herein are incorporated by reference in their entirety. In case of conflict, the present specification, including definitions, will control.

### DETAILED DESCRIPTION

Overhydration in dialysis patients can lead to swelling, discomfort, hypertension, left ventricular hypertrophy, arterial stiffness, atherosclerosis uremic cardiomyopathy, and cardiovascular morbidity. Overhydration is also associated with mortality in chronic kidney disease (see, e.g., Vega et al. Clin Kidney J. 2018; 11(3): 372-376). As such, fluids are typically restricted in dialysis and chronic kidney disease patients, but many patients find it difficult abide by their fluid restrictions and take all of their oral medications. Accordingly, provided herein are compositions including: a vitamin B component; vitamin D; a magnesium component, and optionally a zinc component. In some embodiments, the composition is a liquid, e.g., the composition further includes water.

In some embodiments, the amounts of each component, as provided herein, corresponds to the amount per 32 ounces of a liquid formulation.

In some embodiments, the vitamin B component includes one or more of: folic acid, vitamin B12, vitamin B6, vitamin B1, vitamin B3, and vitamin B5 (e.g., any two, three, four, five, or all six of folic acid, vitamin B12, vitamin B6, vitamin B1, vitamin B3, and vitamin B5).

In some embodiments, the vitamin B component includes folic acid. In some embodiments, the folic acid is present in an amount of about 500 μg to about 1600 μg. For example, about 500 μg to about 600 about 500 μg to about 700 about 500 μg to about 800 about 500 μg to about 900 about 500 μg to about 1000 μg, about 500 μg to about 1100 μs, about 500 μg to about 1200 μg, about 500 μg to about 1300 μg, about 500 μg to about 1400 μg, about 500 μg to about 1500 μg, about 1500 μs to about 1600 μg, about 1400 μg to about 1600 μg, about 1300 μg to about 1600 μg, about 1200 μg to about 1600 μg, about 1100 μg to about 1600 μs, about 1000 μg to about 1600 μg, about 900 μs to about 1600 μg, about 800 μg to about 1600 μs, about 700 μg to about 1600 μg, or about 600 μg to about 1600 μg. In some embodiments, the folic acid is present in an amount of about 750 μg to about 1250 μg. For example, about 750 μg, about 800 μg, about 850 μg, about 900 μs, about 950 μg, about 1000 μs, about 1050 μg, about 1100 μg, about 1150 μg, about 1200 μg, or about 1250 μg.

In some embodiments, the folic acid is present in an amount of about 16 to about 50 μg per ounce of a liquid composition. For example, about 16 to about 20, about 16 to about 30, about 16 to about 40, about 40 to about 50, about 30 to about 50, about 20 to about 50 μs per ounce of a liquid composition. In some embodiments, the folic acid is present in an amount of about 23 to about 40 μg per ounce of a liquid composition. For example, about 23, about 25, about 26, about 27, about 28, about 29, about 30, about 31, about 32, about 33, about 34, about 35, about 36, about 37, about 38, or about 40 μg per ounce of a liquid composition.

In some embodiments, the vitamin B component includes vitamin B₁₂. In some embodiments, the vitamin B₁₂ is present in an amount of about 1 μg to about 10 μg. For example, about 1 μg to about 2 μg, about 1 μg to about 3 μg, about 1 μg to about 4 μg, about 1 μg to about 5 about 1 μg to about 6 about 1 μg to about 7 about 1 μg to about 8 about 1 μg to about 9 about 9 μg to about 10 μg, about 6 μg to about 10 μg, about 7 μg to about 10 μg, about 6 μg to about 10 μg, about 5 μg to about 10 μg, about 4 μg to about 10 μg, about 3 μg to about 10 μg, or about 2 μg to about 10 μg. In some embodiments, the vitamin B₁₂ present in an amount of about 4 μg to about 8 μg. For example, about 4 μg, about 5 μg, or about 6 μg.

In some embodiments, the vitamin B₁₂ is present in an amount of about 0.03 to about 0.3 μg per ounce of a liquid composition. For example, about 0.03 to about 0.05, about 0.03 to about 0.1, about 0.03 to about 0.15, about 0.03 to about 0.2, about 0.03 to about 0.25, about 0.25 to about 0.3, about 0.2 to about 0.3, about 0.15 to about 0.3, about 0.1 to about 0.3, about 0.05 to about 0.3 μg per ounce of a liquid composition. For example, about 0.1 to about 0.25 μg per ounce of a liquid composition. In some embodiments, the vitamin B12 is present in an amount of about 0.1, about 0.15, about 0.2, or about 0.25 μg per ounce of a liquid composition.

In some embodiments, the vitamin B component includes vitamin B₆. In some embodiments, the vitamin B₆ is present in an amount of about 10 mg to about 50 mg. For example, about 10 mg to about 20 mg, about 10 mg to about 30 mg, about 10 mg to about 40 mg, about 40 mg to about 50 mg, or about 30 mg to about 50 mg, or about 20 mg to about 50 mg. In some embodiments, the vitamin B₆ is present in an amount of about 20 mg to about 30 mg. In some embodiments, the vitamin B₆ is present in an amount of about 15 mg, about 20 mg, about 25 mg, about 30 mg, about 35 mg, or about 40 mg.

In some embodiments, the vitamin B₆ is present in an amount of about 310 to about 1560 μg per ounce of a liquid composition. For example, about 310 to about 500, about 310 to about 750, about 310 to about 1000, about 310 to about 1250, about 1250 to about 1560, about 1000 to about 1560, about 750 to about 1560, or about 500 to about 1560 μg per ounce of a liquid composition. In some embodiments, the vitamin B6 is present in an amount of about 625 to about 940 μg per ounce of a liquid composition. For example, about 625, about 650, about 675, about 700, about 725, about 750, about 775, about 800, about 825, about 850, about 875, about 900, or about 925 μg per ounce of a liquid composition.

In some embodiments, the vitamin B component includes vitamin B₁. In some embodiments, the vitamin B₁ is present in an amount of about 0.1 mg to about 4 mg. For example, about 0.1 mg to about 1 mg, about 0.1 mg to about 0.5 mg, about 0.1 mg to about 2 mg, about 0.1 mg to about 3 mg, about 3 mg to about 4 mg, about 2 mg to about 4 mg, about 1 mg to about 4 mg, or about 0.5 mg to about 4 mg. In some embodiments, the vitamin B₁ is present in an amount of about 1 mg to about 2 mg. In some embodiments, the vitamin B₁ is present in an amount of about 0.1 mg, about 0.5 mg, about 1 mg, about 1.5 mg, about 2 mg, about 2.5 mg, about 3 mg, about 3.5 mg, or about 4 mg.

In some embodiments, the vitamin B₁ is present in an amount of about 3 to about 125 μg per ounce of a liquid composition. For example, about 3 to about 20, about 3 to about 40, about 3 to about 60, about 3 to about 80, about 3 to about 100, about 3 to about 120, about 100 to about 125, about 80 to about 125, about 60 to about 125, about 40 to about 125 or about 20 to about 125 μs per ounce of a liquid composition. In some embodiments, the vitamin B₁ is present in an amount of about 3, about 15, about 30, about 45, about 60, about 75, about 90, about 105, or about 120 μg per ounce of a liquid composition.

In some embodiments, the vitamin B component includes vitamin B₃. In some embodiments, the vitamin B₃ is present in an amount of about 10 mg to about 30 mg. For example, about 10 mg to about 15 mg, about 10 mg to about 20 mg, about 10 mg to about 25 mg, about 25 mg to about 30 mg, about 20 mg to about 30 mg, or about 15 mg to about 30 mg. In some embodiments, the vitamin B₃ is present in an amount of about 15 mg to about 25 mg. In some embodiments, the vitamin B₃ is present in an amount of about 10 mg, about 15 mg, about 16 mg, about 17 mg, about 18 mg, about 19 mg, about 20 mg, about 21 mg, about 22 mg, about 23 mg, about 24 mg, about 25 mg, or about 30 mg.

In some embodiments, the vitamin B₃ is present in an amount of about 310 to about 940 μg per ounce of a liquid composition. For example, about 310 to about 400, about 310 to about 500, about 310 to about 600, about 310 to about 700, about 310 to about 800, about 310 to about 900, about 800 to about 900, about 700 to about 900, about 600 to about 900, about 500 to about 900, or about 400 to about 900 μg per ounce of a liquid composition. In some embodiments, the vitamin B₃ is present in an amount of about 310, about 350, about 400, about 450, about 500, about 550, about 600, about 625, about 635, about 650, about 660, about 670, about 680, about 690, about 700, about 710, about 720, about 725, about 730, about 740, about 750, about 760, about 770, about 775, about 780, about 790, about 800, about 850, about 900, or about 940 μs per ounce of a liquid composition.

In some embodiments, the vitamin B component includes vitamin B₅. In some embodiments, the vitamin B₅ is present in an amount of about 5 mg to about 15 mg. For example, 5 mg to about 14 mg, about 5 mg to about 13 mg, about 5 mg to about 12 mg, about 5 mg to about 11 mg, about 5 mg to about 10 mg, about 5 mg to about 9 mg, about 5 mg to about 8 mg, about 5 mg to about 7 mg, about 5 mg to about 6 mg, about 6 mg to about 15 mg, about 7 mg to about 15 mg, about 8 mg to about 15 mg, about 9 mg to about 15 mg, about 10 mg to about 15 mg, about 11 mg to about 15 mg, about 12 mg to about 15 mg, about 13 mg to about 15 mg, or about 14 mg to about 15 mg. In some embodiments, the vitamin B₅ is present in an amount of about 5 mg, about 6 mg, about 7 mg, about 8 mg, about 9 mg, about 10 mg, about 11 mg, about 12 mg, about 13 mg, about 14 mg, or about 15 mg.

In some embodiments, the vitamin B₅ is present in an amount of about 155 to about 470 μg per ounce of a liquid composition. For example, about 155 to about 200, about 155 to about 250, about 155 to about 300, about 155 to about 350, about 155 to about 400, about 155 to about 450, about 450 to about 470, about 400 to about 470, about 350 to about 470, about 300 to about 470, about 250 to about 470, or about 200 to about 470 μg per ounce of a liquid composition.

In some embodiments, the vitamin B component includes biotin. In some embodiments, the biotin is present in an amount of about 50 mg to about 200 mg. For example, about 50 mg to about 175 mg, about 50 mg to about 150 mg, about 50 mg to about 125 mg, about 50 mg to about 100 mg, about 50 mg to about 75 mg, about 75 mg to about 200 mg, about 100 mg to about 200 mg, about 125 mg to about 200 mg, about 150 mg to about 200 mg, or about 175 mg to about 200 mg. In some embodiments, the biotin is present in an amount of about 50 mg, about 75 mg, about 100 mg, about 125 mg, about 150 mg, about 175 mg, or about 200 mg.

In some embodiments, the biotin is present in an amount of about 1550 to about 6250 μg per ounce of a liquid composition. For example, about 1550 to about 2000, about 1550 to about 2500, about 1550 to about 3000, about 1550 to about 3500, about 1550 to about 4000, about 1550 to about 4500, about 1550 to about 5000, about 1550 to about 5500, about 1550 to about 6000, about 6000 to about 6250, about 5500 to about 6250, about 5000 to about 6250, about 4500 to about 6250, about 4000 to about 6250, about 3500 to about 6250, about 3000 to about 6250, about 2500 to about 6250, or about 2000 to about 6250 μg per ounce of a liquid composition. In some embodiments, the biotin is present in an amount of about 1550, about 2350, about 3125, about 3900, about 4690, about 5470, or about 6250 μg per ounce of a liquid composition.

In some embodiments, the vitamin B component includes at least two of folic acid, vitamin B₁₂, vitamin B₆, vitamin B₁, vitamin B₃, and vitamin B₅. In some embodiments, the vitamin B component includes at least three of folic acid, vitamin B₁₂, vitamin B₆, vitamin B₁, vitamin B₃, and vitamin B₅. In some embodiments, the vitamin B component includes at least four of folic acid, vitamin B₁₂, vitamin B₆, vitamin B₁, vitamin B₃, and vitamin B₅. In some embodiments, the vitamin B component includes folic acid, vitamin B₁₂, vitamin B₆, vitamin B₁, vitamin B₃, and vitamin B₅.

In some embodiments, the vitamin D is present in an amount of about 4,000 IU to about 10,000 IU. For example, about 4,000 IU to about 9,000 IU, about 4,000 IU to about 8,000 IU, about 4,000 IU to about 7,000 IU, about 4,000 IU to about 6,000 IU, about 4,000 IU to about 5,000 IU, about 5,000 IU to about 10,000 IU, about 6,000 IU to about 10,000 IU, about 7,000 IU to about 10,000 IU, about 8,000 IU to about 10,000 IU, or about 9,000 IU to about 10,000 IU. In some embodiments, the vitamin D is present in an amount of about 4,000 IU, about 5,000 IU, about 6,000 IU, about 7,000 IU, about 8,000 IU, about 9,000 IU, or about 10,000 IU. In some embodiments, the vitamin D is present in an amount of about 5,000 IU.

In some embodiments, the vitamin D is present in an amount of about 125 to about 315 IU per ounce of a liquid composition. For example, about 125 to about 150, about 125 to about 175, about 125 to about 200, about 125 to about 225, about 125 to about 250, about 125 to about 275, about 125 to about 300, about 300 to about 315, about 275 to about 315, about 250 to about 315, about 225 to about 315, about 200 to about 315, about 175 to about 315, or about 150 to about 315 IU per ounce of a liquid composition. In some embodiments, the vitamin D is present in an amount of about 125, about 150, about 175, about 200, about 225, about 250, about 275, about 300, or about 315 IU per ounce of a liquid composition.

In some embodiments, the magnesium component includes magnesium carbonate, magnesium oxide, magnesium chloride, magnesium citrate, magnesium sulfate, magnesium gluconate, magnesium hydroxide, or a combination thereof.

In some embodiments, the magnesium component includes magnesium carbonate. In some embodiments, the magnesium carbonate is present in an amount of about 100 mg to about 200 mg. For example, about 100 mg to about 175 mg, about 100 mg to about 150 mg, about 100 mg to about 125 mg, about 125 mg to about 200 mg, about 150 mg to about 200 mg, or about 175 mg to about 200 mg. In some embodiments, the magnesium carbonate is present in an amount of about 100 mg, about 125 mg, about 150 mg, about 175 mg, or about 200 mg.

In some embodiments, the magnesium carbonate is present in an amount of about 3 to about 6.5 mg per ounce of a liquid composition. For example, about 3 to about 3.5, about 3 to about 4, about 3 to about 4.5, about 3 to about 5, about 3 to about 5.5, about 3 to about 6, about 6 to about 6.5, about 5.5 to about 6.5, about 5 to about 6.5, about 4.5 to about 6.5, about 4 to about 6.5, or about 3.5 to about 6.5 mg per ounce of a liquid composition. In some embodiments, the magnesium carbonate is present in an amount of about 3, about 3.5, about 4, about 4.5, about 5, about 5.5, about 6, or about 6.5 mg per ounce of a liquid composition.

In some embodiments, the magnesium component includes magnesium oxide. In some embodiments, the magnesium oxide is present in an amount of about 200 mg to about 500 mg. For example, about 200 mg to about 450 mg, about 200 mg to about 400 mg, about 200 mg to about 350 mg, about 200 mg to about 300 mg, about 200 mg to about 250 mg, about 250 mg to about 500 mg, about 300 mg to about 500 mg, about 350 mg to about 500 mg, about 400 mg to about 500 mg, or about 450 mg to about 500 mg. In some embodiments, the magnesium oxide is present in an amount of about 200 mg, about 250 mg, about 300 mg, about 350 mg, about 400 mg, about 450 mg, or about 500 mg. In some embodiments, magnesium oxide present in an amount of about 400 mg.

In some embodiments, the magnesium carbonate is present in an amount of about 6 to about 16 mg per ounce of a liquid composition. For example, about 6 to about 7, about 6 to about 8, about 6 to about 9, about 6 to about 10, about 6 to about 11, about 6 to about 12, about 6 to about 13, about 6 to about 14, about 6 to about 15, about 15 to about 16, about 14 to about 16, about 13 to about 16, about 12 to about 16, about 11 to about 16, about 10 to about 16, about 9 to about 16, about 8 to about 16, or about 7 to about 16 mg per ounce of a liquid composition. In some embodiments, the magnesium carbonate is present in an amount of about 6, about 7, about 8, about 9, about 10, about 11, about 12, about 13, about 14, about 15, or about 16 mg per ounce of a liquid composition.

In some embodiments, the composition further includes zinc. As used herein, “zinc” can refer to elemental zinc and zinc salts such as zinc gluconate, zinc sulfate, zinc acetate, zinc citrate, zinc bisglycinate, zinc oxide, zinc aspartate, zinc monomethionine, and zinc picolinate. In some embodiments, “zinc” refers to any form of zinc suitable for use as a human supplement. In some embodiments, “zinc” refers to combinations of elemental zinc and one or more zinc salts. In some embodiments, “zinc” refers to combinations of two or more zinc salts.

In some embodiments, the zinc is present in an amount of about 25 mg to about 100 mg. For example, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 25 mg to about 30 mg, about 30 mg to about 100 mg, about 35 mg to about 100 mg, about 40 mg to about 100 mg, about 45 mg to about 100 mg, about 50 mg to about 100 mg, about 55 mg to about 100 mg, about 60 mg to about 100 mg, about 65 mg to about 100 mg, about 70 mg to about 100 mg, about 75 mg to about 100 mg, about 80 mg to about 100 mg, about 85 mg to about 100 mg, about 90 mg to about 100 mg, or about 95 mg to about 100 mg. In some embodiments, the zinc is present in an amount of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, the zinc is present in an amount of about 50 mg.

In some embodiments, the zinc is present in an amount of about 0.7 to about 3.5 mg per ounce of a liquid composition. For example, about 0.7 to about 1, about 0.7 to about 1.5, about 0.7 to about 1.75, about 0.7 to about 2, about 0.7 to about 2.25, about 0.7 to about 2.5, about 0.7 to about 2.75, about 0.7 to about 3, about 0.7 to about 3.25, about 3.25 to about 3.5, about 3 to about 3.5, about 2.75 to about 3.5, about 2.5 to about 3.5, about 2.25 to about 3.5, about 2 to about 3.5, about 1.5 to about 3.5, or about 1 to about 3.5 mg per ounce of a liquid composition. In some embodiments, the zinc is present in an amount of about 0.7, about 1, about 1.25, about 1.5, about 1.75, about 2, about 2.25, about 2.5, about 2.75, about 3, about 3.25, or about 3.5 mg per ounce of a liquid composition.

In some embodiments, the composition further includes one or more phosphorous binders. Non-limiting examples of a phosphorous binder include: sevelamer HCl, lanthanum carbonate, sucroferric oxyhydroxide, and ferric citrate.

In some embodiments, the one or more phosphorous binders include sevelamer HCl. In some embodiments, the sevelamer HCl is present in an amount of about 2000 mg to about 8000 mg. For example, about 2000 mg to about 7000 mg, about 2000 mg to about 6000 mg, about 2000 mg to about 5000 mg, about 2000 mg to about 4000 mg, about 2000 mg to about 3000 mg, about 3000 mg to about 8000 mg, about 4000 mg to about 8000 mg, about 5000 mg to about 8000 mg, about 6000 mg to about 8000 mg, or about 7000 mg to about 8000 mg. In some embodiments, the sevelamer HCl is present in an amount of about 2000 mg, about 2200 mg, about 2400 mg, about 2600 mg, about 2800 mg, about 3000 mg, about 3200 mg, about 3400 mg, about 3600 mg, about 3800 mg, about 4000 mg, about 4200 mg, about 4400 mg, about 4600 mg, about 4800 mg, about 5000 mg, about 5200 mg, about 5400 mg, about 5600 mg, about 5800 mg, about 6000 mg, about 6200 mg, about 6400 mg, about 6600 mg, about 6800 mg, about 7000 mg, about 7200 mg, about 7400 mg, about 7600 mg, about 7800, or about 8000 mg.

In some embodiments, the sevelamer HCl is present in an amount of about 60 to about 250 mg per ounce of a liquid composition. For example, about 60 to about 100, about 60 to about 150, about 60 to about 200, about 200 to about 250, about 150 to about 250, or about 100 to about 250 mg per ounce of a liquid composition. In some embodiments, the sevelamer HCl is present in an amount of about 60, about 100, about 125, about 150, about 175, about 200, about 225, or about 250 mg per ounce of a liquid composition.

In some embodiments, the one or more phosphorous binders include lanthanum carbonate. In some embodiments, the lanthanum carbonate is present in an amount of about 1000 mg to about 3500 mg. For example, about 1000 mg to about 3000 mg, about 1000 mg to about 2500 mg, about 1000 mg to about 2000 mg, about 1000 mg to about 1500 mg, about 1500 mg to about 3500 mg, about 2000 mg to about 3500 mg, about 2500 mg to about 3500 mg, or about 3000 mg to about 3500 mg. In some embodiments, the lanthanum carbonate is present in an amount of about 1000 mg, about 1500 mg, about 2000 mg, about 2500 mg, about 3000 mg, or about 3500 mg.

In some embodiments, the lanthanum carbonate is present in an amount of about 30 to about 110 mg per ounce of a liquid composition. For example, about 30 to about 40, about 30 to about 50, about 30 to about 60, about 30 to about 70, about 30 to about 80, about 30 to about 90, about 30 to about 100, about 100 to about 110, about 90 to about 110, about 80 to about 110, about 70 to about 110, about 60 to about 110, about 50 to about 110, or about 40 to about 110 mg per ounce of a liquid composition. In some embodiments, the lanthanum carbonate is present in an amount of about 30, about 40, about 50, about 60, about 70, about 80, about 90, about 110, or about 110 mg per ounce of a liquid composition.

In some embodiments, the one or more phosphorous binders include sucroferric oxyhydroxide. In some embodiments, the sucroferric oxyhydroxide is present in an amount of about 1000 mg to about 3500 mg. For example, about 1000 mg to about 3000 mg, about 1000 mg to about 2500 mg, about 1000 mg to about 2000 mg, about 1000 mg to about 1500 mg, about 1500 mg to about 3500 mg, about 2000 mg to about 3500 mg, about 2500 mg to about 3500 mg, or about 3000 mg to about 3500 mg. In some embodiments, the sucroferric oxyhydroxide is present in an amount of about 1000 mg, about 1500 mg, about 2000 mg, about 2500 mg, about 3000 mg, or about 3500 mg.

In some embodiments, the sucroferric oxyhydroxide is present in an amount of about 30 to about 110 mg per ounce of a liquid composition. For example, about 30 to about 40, about 30 to about 50, about 30 to about 60, about 30 to about 70, about 30 to about 80, about 30 to about 90, about 30 to about 100, about 100 to about 110, about 90 to about 110, about 80 to about 110, about 70 to about 110, about 60 to about 110, about 50 to about 110, or about 40 to about 110 mg per ounce of a liquid composition. In some embodiments, the sucroferric oxyhydroxide is present in an amount of about 30, about 40, about 50, about 60, about 70, about 80, about 90, about 110, or about 110 mg per ounce of a liquid composition.

In some embodiments, the one or more phosphorous binders include ferric citrate. In some embodiments, the ferric citrate is present in an amount of about 550 mg to about 3000 mg. For example, about 550 mg to about 2500 mg, about 550 mg to about 2000 mg, about 550 mg to about 1500 mg, about 550 mg to about 1000 mg, about 550 mg to about 750 mg, about 750 mg to about 3000 mg, about 1000 mg to about 3000 mg, about 1500 mg to about 3000 mg, about 2000 mg to about 3000 mg, about 2500 mg to about 3000 mg, or about 3000 mg to about 3000 mg. In some embodiments, the sucroferric oxyhydroxide is present in an amount of about 550 mg, about 600 mg, about 630 mg, about 650 mg, about 675 mg, about 700 mg, about 750 mg, about 800 mg, about 850 mg, about 900 mg, about 950 mg, about 1000 mg, about 1050 mg, about 1100 mg, about 1150 mg, about 1200 mg, about 1240 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1890 mg, about 1900 mg, about 1950 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2520 mg, about 2550 mg, about 3000 mg, about 3100 mg, about 3200 mg, about 3300 mg, about 3400 mg, or about 3500 mg.

In some embodiments, the ferric citrate is present in an amount of about 17 to about 95 mg per ounce of a liquid composition. For example, about 17 to about 20, about 17 to about 30, about 17 to about 40, about 17 to about 50, about 17 to about 60, about 17 to about 70, about 17 to about 80, about 17 to about 90, about 90 to about 95, about 80 to about 95, about 70 to about 95, about 60 to about 95, about 50 to about 95, about 40 to about 95, about 30 to about 95, or about 20 to about 95 mg per ounce of a liquid composition. In some embodiments, the ferric citrate is present in an amount of about 17, about 20, about 25, about 30, about 35, about 40, about 45, about 50, about 55, about 60, about 65, about 70, about 75, about 80, about 85, about 90, or about 95 mg per ounce of a liquid composition.

In some embodiments, wherein the composition further includes water, the water is provided in an amount such that the final volume of the composition is 32 ounces.

In some embodiments, the composition further includes a buffer.

In some embodiments, the composition does not include one or more of: a protein component, vitamin C, vitamin A, vitamin K, vitamin E, iron, calcium, and an omega-3 fatty acid. In some embodiments, the composition does not include: a protein, vitamin C, vitamin A, vitamin K, vitamin E, iron, calcium, and an omega-3 fatty acid. In some embodiments, the composition does not include: a protein, vitamin C, vitamin A, vitamin K, vitamin E, calcium, and an omega-3 fatty acid.

In some embodiments, the composition does not include sodium chloride. In some embodiments, the composition does not include a sugar or sweetener.

In some embodiments, the composition includes: a vitamin B component; vitamin D present in an amount of about 4,000 IU to about 10,000 IU; and a magnesium component.

In some embodiments, the vitamin B component includes folic acid present in an amount of about 500 μg to about 1600 μg. In some embodiments, the vitamin B component includes one or more of: folic acid present in an amount of about 500 μg to about 1600 μg; vitamin B₁₂ present in an amount of about 1 μg to about 10 μg; vitamin B₆ present in an amount of about 10 mg to about 50 mg; vitamin B₁ present in an amount of about 1 mg to about 2 mg; vitamin B₃ present in an amount of about 15 mg to about 25 mg; vitamin B₅ present in an amount of about 5 mg to about 15 mg; and biotin present in an amount of about 50 mg to about 200 mg.

In some embodiments, the vitamin B component includes folic acid present in an amount of about 1000 μg. In some embodiments, the vitamin B component includes one or more of: folic acid present in an amount of about 1000 μg; vitamin B₁₂ present in an amount of about 6 μg; vitamin B₆ present in an amount of about 25 mg; vitamin B₁ present in an amount of about 1.5 mg; vitamin B₃ present in an amount of about 20 mg; vitamin B₅ present in an amount of about 10 mg; and biotin present in an amount of about 100 mg.

In some embodiments, the magnesium component includes: magnesium carbonate present in an amount of about 100 mg to about 200 mg; magnesium oxide present in an amount of about 200 mg to about 500 mg; or a combination thereof.

In some embodiments, the composition further includes one or more phosphorous binders. In some embodiments, the one or more phosphorous binders is selected from: sevelamer HCl present in an amount of about 2000 mg to about 8000 mg; lanthanum carbonate present in an amount of about 1000 mg to about 3500 mg; sucroferric oxyhydroxide present in an amount of about 1000 mg to about 3500 mg; or ferric citrate present in an amount of about 550 mg to about 3000 mg.

In some embodiments, the composition includes: folic acid present in an amount of about 500 μg to about 1600 μg; vitamin D present in an amount of about 4,000 IU to about 10,000 IU; and magnesium carbonate present in an amount of about 100 mg to about 200 mg.

In some embodiments, the composition further includes one or more of: vitamin B₁₂ present in an amount of about 1 μg to about 10 μg; vitamin B₆ present in an amount of about 10 mg to about 50 mg; vitamin B₁ present in an amount of about 1 mg to about 2 mg; vitamin B₃ present in an amount of about 15 mg to about 25 mg; vitamin B₅ present in an amount of about 5 mg to about 15 mg; and biotin present in an amount of about 50 mg to about 200 mg. In some embodiments, the vitamin B₁₂ is present in an amount of about 6 μg. In some embodiments, the vitamin B₆ present in an amount of about 25 mg. In some embodiments, the vitamin B₁ present in an amount of about 1.5 mg. In some embodiments, the vitamin B₃ present in an amount of about 20 mg. In some embodiments, the vitamin B₅ present in an amount of about 10 mg. In some embodiments, the biotin present in an amount of about 100 mg.

In some embodiments, the composition further includes one or more phosphorous binders. In some embodiments, the one or more phosphorous binders is selected from: sevelamer HCl present in an amount of about 2000 mg to about 8000 mg; lanthanum carbonate present in an amount of about 1000 mg to about 3500 mg; sucroferric oxyhydroxide present in an amount of about 1000 mg to about 3500 mg; or ferric citrate present in an amount of about 550 mg to about 3000 mg.

In some embodiments, the composition includes: folic acid present in an amount of about 1000 μg; vitamin D present in an amount of about 5,000 IU; and magnesium carbonate present in an amount of about 100 mg to about 200 mg. In some embodiments, the composition further includes one or more of: vitamin B₁₂ present in an amount of about 1 μg to about 10 μg; vitamin B₆ present in an amount of about 10 mg to about 50 mg; vitamin B₁ present in an amount of about 1 mg to about 2 mg; vitamin B₃ present in an amount of about 15 mg to about 25 mg; vitamin B₅ present in an amount of about 5 mg to about 15 mg; and biotin present in an amount of about 50 mg to about 200 mg. In some embodiments, the vitamin B₁₂ is present in an amount of about 6 μg. In some embodiments, the vitamin B₆ present in an amount of about 25 mg. In some embodiments, the vitamin B₁ present in an amount of about 1.5 mg. In some embodiments, the vitamin B₃ present in an amount of about 20 mg. In some embodiments, the vitamin B₅ present in an amount of about 10 mg. In some embodiments, the biotin present in an amount of about 100 mg.

In some embodiments, the composition further includes one or more phosphorous binders. In some embodiments, the one or more phosphorous binders is selected from: sevelamer HCl present in an amount of about 2000 mg to about 8000 mg; lanthanum carbonate present in an amount of about 1000 mg to about 3500 mg; sucroferric oxyhydroxide present in an amount of about 1000 mg to about 3500 mg; or ferric citrate present in an amount of about 550 mg to about 3000 mg.

In some embodiments, a composition as described herein is formulated for oral delivery. A composition as described herein can be formulated for oral delivery in a variety of ways. For example, the composition can be in the form of a tablet or powder. In some embodiments, the tablet or powder can be reconstituted in water prior to delivery to the subject. As another example, a composition as described herein can be in the form of a liquid, solution, or suspension.

Liquid preparations for oral administration can take the form of, for example, solutions, syrups, or suspension, or they can be presented as a dry product for constitution with water or other suitable liquid vehicle before use. For example, a composition as described herein can be presented as dry powder and dissolved in a suitable liquid carrier. In some embodiments, a composition as described herein can be diluted in a suitable liquid carrier.

Tablets and powders can be configured to have a unit dosage equal to the daily desired dosage. For example, if a subject desires 1000 mg of a particular composition, each tablet can be 1000 mg in weight. As another example, if a subject desires 1000 mg of a particular composition each tablet can be 500 mg in weight and the subject can take two tablets. The dosages of a particular composition will depend on many factors including the mode of administration. As an example, a composition as described herein can be formulated in a dose such that an individual receives the amounts as shown in Table 1, e.g., in a liquid formulation.

Also provided herein are methods for preventing overhydration in a patient in need thereof including administering any of the compositions described herein.

In some embodiments, the composition is a powder. In some embodiments, the powder is added to about 32 ounces of water prior to administration.

In some embodiments, the composition is a liquid. For example, the composition is prepackaged in a ready-to-use liquid formulation (e.g., in 32-ounce bottles for daily administration). In some embodiments, the composition may be packaged as a concentrated liquid solution to be diluted prior to administration (e.g., diluted into about 32-ounces of liquid for daily administration).

In some embodiments, the liquid composition is prepackaged in a 32-ounce bottle comprising an amount of each component as provided herein. In some embodiments, the liquid composition is prepackaged in bottles less than or greater than about 32 ounces and the amounts of each component as described herein is provided in a corresponding amount. For example, if the prepackaged bottle is about 3×, about 2×, about 0.75×, about 0.5×, or about 0.25× of 32 ounces (e.g., about 96 ounces, about 64 ounces, about 24 ounces, about 16 ounces, or about 8 ounces, respectively) then the amount of each component is about about 3×, about 2×, about 0.75×, about 0.5×, or about 0.25× of the amount of each component as described herein.

In some embodiments, the composition is administered once daily. In some embodiments, the composition is administered once daily for a period of about two days to about 5 years. In some embodiments, the composition is administered once daily for a period of about two days to about 1 month, about two days to about 6 months, about 2 days to about 1 year, about 2 days to about 2.5 years, about 2.5 years to about 5 years, about 1 year to about 5 years, about 6 months to about 5 years, or about 1 month to about 5 years.

In some embodiments, the patient is on dialysis. In some embodiments, the patient has chronic kidney disease.

### EXAMPLES

The invention will be described in greater detail by way of specific examples. The following examples are offered for illustrative purposes, and to exemplify the topical compositions and methods described herein and are not intended to limit the invention in any manner. Many variations will suggest themselves and are within the full intended scope. Those of skill in the art will readily recognize a variety of non-critical parameters that can be changed or modified to yield essentially the same results.

### Example 1

A composition is prepared as shown in Table 1.

TABLE 1

| | Amount (per 32 |
|---|---|
| Ingredient | oz of solution) |
| Folic acid | 1 mg |
| Vitamin B12 | 6 μg |
| Vitamin B6 | 25 mg |
| Vitamin B3 | 20 mg |
| Vitamin B5 | 10 mg |
| Biotin | 100 mg |
| Zinc | 50 mg |
| Magnesium Oxide | 400 mg |
| Sevelamer HCl | 2400 mg |

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

1. A composition comprising:
a vitamin B component;
vitamin D;
a magnesium component; and
water.

2. The composition of claim 1, wherein the vitamin B component comprises one or more of: folic acid, vitamin B₁₂, vitamin B₆, vitamin B₁, vitamin B₃, and vitamin B₅.

3. The composition of claim 1, wherein the vitamin B component comprises one or more of:
folic acid present in an amount of about 500 μg to about 1600 μg;
vitamin B₁₂ present in an amount of about 1 μg to about 10 μg;
vitamin B₆ present in an amount of about 10 mg to about 50 mg;
vitamin B₁ present in an amount of about 1 mg to about 2 mg;
vitamin B₃ present in an amount of about 15 mg to about 25 mg;
vitamin B₅ present in an amount of about 5 mg to about 15 mg; and
biotin present in an amount of about 50 mg to about 200 mg.

4.-10. (canceled)

11. The composition of claim 1, wherein the vitamin B component comprises folic acid, vitamin B₁₂, vitamin B₆, vitamin B₁, vitamin B₃, and vitamin B₅.

12. The composition of claim 1, wherein the vitamin D is present in an amount of about 4,000 IU to about 10,000 IU.

13. (canceled)

14. The composition of claim 1, wherein the magnesium component comprises magnesium carbonate, magnesium oxide, magnesium chloride, magnesium citrate, magnesium sulfate, magnesium gluconate, magnesium hydroxide, or a combination thereof.

15. The composition of claim 14, wherein the magnesium component comprises
magnesium carbonate present in an amount of about 100 mg to about 200 mg;
magnesium oxide present in an amount of about 200 mg to about 500 mg; or
a combination thereof.

16. (canceled)

17. The composition of claim 1, wherein the composition comprises:
folic acid present in an amount of about 1000 μg;
vitamin D is present in an amount of about 5,000 IU; and
magnesium carbonate present in an amount of about 100 mg to about 200 mg.

18. The composition of claim 1, wherein the composition further comprises zinc.

19. The composition of claim 18, wherein the zinc is present in an amount of about 25 mg to about 100 mg.

20. (canceled)

21. The composition of claim 1, wherein the composition further comprises one or more phosphorous binders.

22. The composition of claim 21, wherein the one or more phosphorous binders comprise: sevelamer HCl, lanthanum carbonate, sucroferric oxyhydroxide, or ferric citrate.

23. The composition of claim 22, wherein the one or more phosphorous binders comprises:
sevelamer HCl present in an amount of about 2000 mg to about 8000 mg;
lanthanum carbonate present in an amount of about 1000 mg to about 3500 mg;
sucroferric oxyhydroxide present in an amount of about 1000 mg to about 3500 mg;
ferric citrate present in an amount of about 550 mg to about 3000 mg; or
a combination thereof.

24.-34. (canceled)

35. The composition of claim 1, wherein the composition does not comprise one or more of: a protein component, vitamin C, vitamin A, vitamin K, vitamin E, iron, calcium, and an omega-3 fatty acid.

36. The composition of claim 35, wherein the composition does not comprise: a protein, vitamin C, vitamin A, vitamin K, vitamin E, calcium, and an omega-3 fatty acid.

37. The composition of claim 1, wherein the composition further comprises a buffer.

38. The composition of claim 1, wherein the composition does not comprise sodium chloride.

39. The composition of claim 1, wherein the composition does not include a sugar or sweetener.

40. A method for reducing overhydration in a patient in need thereof, the method comprising administering a composition of claim 1.

41. (canceled)
